(19) United States
(12) Patent Application Publication
(10) Pub. No.: US 2024/0197211 A1
(43) Pub. Date: Jun. 20, 2024

(54) SYSTEM AND METHOD FOR BLOOD GLUCOSE MONITORING BASED ON HEART RATE VARIABILITY

(71) Applicant: INNOVATION DOOEL, Skopje (MK)

(72) Inventor: Marjan GUSHEV, Skopje (MK)

(73) Assignee: INNOVATION DOOEL, Skopje (MK)

(21) Appl. No.: 18/279,113

(22) PCT Filed: Dec. 29, 2020
(86) PCT No.: PCT/IB2020/062506

§ 371 Date: Aug. 28, 2023

(51) Int. Cl. A61B 5/145; A61B 5/00; A61B 5/352

(52) U.S. Cl. CPC A61B 5/14532; A61B 5/352; A61B 5/7221

(57) ABSTRACT

The concept of this invention is a method to calculate the glucose management index based on calculated instantaneous plasma blood glucose levels from heart rate variability (HRV) parameters received by a QRS detector and ECG sensor. The invented method starts with the extraction of clean electrocardiogram intervals, and then calculates an aggregation of HRV collections satisfying the coverage factor to reveal the ability to control the glucose and quantitative glucose level from long-term HRV; and ambulatory glucose profile along with other statistical glycemic measures by calculating plasma glucose levels from selected short term HRV. The patient, caregiver and doctor can access this glucose profile remotely by a smartphone or cloudbased processing and monitoring system, which can send out an emotionally intelligent message to the user without generating stress, but instead of giving gentle advice and tips for current glycemic state helping to undertake appropriate therapeutic measures.

### BACKGROUND OF THE INVENTION

### Technical Field

The present invention relates to monitoring and estimating blood and plasma glucose levels with an electrocardiogram (ECG) sensor and an information-processing device.

### State of the Art

Many blood glucose-monitoring systems have been invented, developed and marketed over the years with a goal to provide to the patient a higher accuracy in the assessment of a blood glucose level, and almost all use invasive or minimally invasive techniques. The non-invasive methods are mainly based on spectroscopy methods that lack the required precision and need frequent calibrations.

HRV calculation methods from an ECG segment eliminate the ectopic supraventricular and ventricular beats (Camm et al., 1996) and analyze NN intervals. Some solutions include a method for artifact correction based on threshold and differences from RR intervals to separate ectopic and misplaced beats from normal sinus rhythm, or detecting missed or extra beats (Tarvainen and Niskanen, 2012). De-trending methods are introduced to detect and eliminate slow non-stationary segments (Litvack et al., 1995), (Mitov, 1998).

HRV parameters are classified in at least the following domains: time, frequency, and other domains (Camm, 1996), and can be calculated from ECG measurements as long-term (24 h), short-term (5 min), and ultra-short-term measurements (less than 5 min) as reported in (Shaffer and Ginsberg, 2017), (Baek, et al., 2015), (Kuusela, 2013). The overall conclusion is that long-term HRV variability is more sensitive for detecting diabetes autonomic neuropathy from conventional short-term measures (Maser and Lenhard, 2005), (Camm, 1996).

A big correlation between the HRV from one side and glucose level from the other side has been reported by (Ballinger et al., 2018), (Kudat et al., 2006), (Bellavere, 1995) analyzing that diabetes caused progressive autonomic dysfunction and decreased variability in the heart rate (Maser and Lenhard, 2005), (Meyer et al., 2004).

Several measures are used to express the ability to control the glucose level besides the HbA1C value. GMI, also known as eA1c (Johnson et al., 2019) is calculated from a formula derived from the regression line computed from a plot of mean glucose concentration points on the x-axis and contemporaneously measured HbA1C values on the y-axis (Bergenstal et al., 2018); and in minimally invasive continuous glucose measurement systems is expressed by GMI (%)=3.38+0.02345*[mean glucose in mg/dL] (Beck et al., 2017) or with small variations on the coefficients in the mathematical expression (Bergenstal et al., 2018) that depend on the used dataset. The use of Ambulatory Glucose Profile (AGP) for CGM report was analyzed in several research papers (Bergenstal et al., 2013), (Battelino et al., 2019), (Johnson et al., 2019).

Relative contributions of fasting and postprandial glucose differ according to the level of overall glycemic control (Monnier et al., 2003). Glucose variability can be easily measured and validated by the standard deviation (Frontoni et al., 2013): or by CV (DeVries, 2013), (Rodbard, 2011). The presence of CV in CGM was reported for autonomic neuropathy for inadequately controlled type 2 diabetes patients (Jun et al., 2015). A computer program was released to measure MAGE for CGM with at least 48 h measurements (Fritzsche, 2011). With the following formula MAGE=sum(RAGE/n where (n=number of glycemic excursions>1 SD). IQR as a measure of glucose profile was analyzed by (Rodbard, 2009) and compared to other measures (Rodbard, 2012).

Unavailability to detect the heartbeat type is also another reason why devices implementing Photoplethysmography (PPG) for heart rate detection cannot implement this method, although they use a low cost and non-invasive method based on a simple optical technique to detect volumetric changes in blood in peripheral circulation.

Existing methods for detection of heart rate variability were developed mostly for short-term ECG measurements of patients in supine and resting position by multichannel ambulatory medical devices. Even the research performed on long-term ECG measurements also addressed hospitalized patients, a lot of them in supine and resting position: or measured by a multi-channel Holter device. Wearable ECG sensors introduce new issues and challenges, including:
1) measurements are mostly single-channel ECGs,
2) sampling rate and resolution are smaller than in standard ambulatory medical devices, and
3) muscle noise due to free physical movement and loose contact of electrodes can corrupt the signal significantly.

The presence of noise, single-channel measurements with a smaller sampling rate and resolution prevent correct beat detection and classification, and therefore imprecise HRV calculation.

### SUMMARY OF THE INVENTION

The present invention discloses a method for calculation of heart rate variability from measured ECG and determination of the ability to regulate blood glucose level expressed by the Glucose Management Index (GMI) equivalent to the HbA1C, and builds the AGP based on detections of plasma glucose levels. Overall, the invention relates to a method that calculates heart rate variability and detects the ability of glucose regulation and glucose levels by calculations and threshold comparison.

This invention relies on the fact that the same autonomous nerve system controls the heart rate and regulates the blood glucose level. The invention relates to system and method that may be implemented by wireless remote real-time continuous non-invasive heart monitoring systems based on wearable ECG sensors for non-hospitalized patients at their home and working environment; and describes algorithms that detect the instantaneous daily blood glucose levels or a two-month average of glucose regulation ability by HbA1C using non-invasive methods.

Our invention addresses most of the challenges and builds methods for more accurate calculation of HRV to be used for estimation of glucose levels. Our approach improves HRV calculation, optimized for wearable ECG sensors, specifying the most efficient method of estimation of glucose levels, and enabling continuous analysis, real-time monitoring, and alerting.

The invention enables a caregiver, doctor, and patient to monitor the ability continuously and remotely for real-time blood glucose level control. In addition, this invention describes a method that alerts in case of detecting abnormal low or high blood glucose levels, so a patient can be alerted to take care and measures to prevent dangerous health situations.

### DESCRIPTION OF THE DRAWINGS

Features and advantages of this invention will become apparent from the detailed description of one embodiment of the invention in conjunction with the accompanying drawings, wherein:

FIG. 1 shows a system for blood glucose monitoring.

FIG. 2 shows a method for blood glucose monitoring.

FIG. 3 shows a method for extracting clean ECG signals.

FIG. 4 shows a method for calculating HRV parameters as an aggregation of HRV collections.

FIG. 5 shows a method for calculating glucose regulation ability and GMI.

FIG. 6 shows a method for calculating the set of detected plasma glucose levels and a set of metrics that determine the AGP, including GV, TIR, TAR TBR, and MAGE from the aggregation of HRV collections.

FIG. 7 is an illustration of two normal beats and characteristic waves, including the QRS complex, P, T, and U waves, and how RR interval is calculated as distance between two consecutive beats.

FIG. 8 is an illustration of dirty beats that include supraventricular or atrial (A) beats, ventricular (V) beats, artifacts, and noise.

FIG. 9 is an illustration of marking dirty intervals and extracting “clean ECG intervals” in the ECG strip.

FIG. 10 is an illustration of marking dirty intervals in case of large discrepancies between consecutive RR intervals between N beats.

### DETAILED DESCRIPTION OF THE INVENTION

### Detection of Heartbeats

An ECG is electric presentation of the heart function (FIG. 7), where each beat is characterized by a QRS complex, P and T waves, and in some measurements by U-wave that follows the T-wave. The heart rate is determined by the interval between neighboring beats, usually known as a beat-to-beat, also known as RR interval. HRV is calculated as a measure of the rhythm change, determining if the rhythm is regular, or whether the irregularity is regular (repetitive) or irregular (stochastic). A heartbeat can belong to one of the following classes (AAMI EC57 and IEC 60601-2-47):
N beat class represents a category that includes a normal beat (N), a left bundle branch block beat (L), a right bundle branch block beat (R), or a bundle branch block beat (B) that does not fall into the S, V, F, or Q categories described below;
S beat class contains a supraventricular ectopic beat (SVEB), a supraventricular escape beat (n), an atrial premature beat (A), an atrial escape beat (e), a nodal (junctional) premature beat (J), a nodal (junctional) escape beat (j), or an aberrated atrial premature beat (a);
V beat class includes a ventricular ectopic beat (VEB); a ventricular premature beat (V), an R-on-T ventricular premature beat (r), or a ventricular escape beat (E);
F beat class is specified by a fusion of a ventricular (V) and a normal beat (N);
Q beat class consists of a paced beat (P), a fusion of a paced and a normal beat (f), or a beat that cannot be classified (U);

An ECG can be corrupted by noise caused by muscles or surrounding environment and the QRS detection may classify an artifact (|) that is a signal that looks like a beat but does not originate from the heart.

A digital QRS detector is applied to detect heartbeats, processing the array of ECG samples (obtained by analog to digital conversion of the ECG signal) and resulting in an array of ECG annotations. Each ECG annotation identifies the location (sample ID) of the annotation and the beat type. In addition, a beat type can be a special annotation used for rhythm episode identification, noise, or ST-segment elevation or depression.

### HRV Parameters

Standard HRV parameters analyzed here are calculated as:
SDNN The standard deviation of all NN intervals (expressed in milliseconds).
ASDNN The average of the standard deviation of all NN intervals for all 5-minute segments within the defined time period (expressed in milliseconds).
SDANN The standard deviation of all average NN intervals for all 5-minute segments within the defined time period (expressed in milliseconds).
NN50 Number of successive NN intervals whose difference is >50 ms.
pNN50 The ratio between NN50 and all NN in the defined time period (expressed in per mille (parts per thousand)).
rMSSD Root mean square of successive NN interval differences (expressed in milliseconds).
SD1 Measure of the standard deviation of RR interval variability derived from the RR Poincare plot (expressed in milliseconds).
SD2 Measure of the RR interval variability derived from RR Poincare plot (expressed in milliseconds).
SD1/SD2 The ratio between SD1 and SD2. In addition to the standard HRV parameters we calculate the following HRV parameters applying the NN rule to eliminate those RR intervals, which differ more than 15%.

Most of the research on HRV includes analysis of long-term (24 h) ECGs measured by Holter devices, or by a short-term (5 minutes) or ultra-short term (up to 5 minutes) ECGs measured by ambulatory medical devices.

### Statistical Methods

Variability of quantitative and continuous data can be expressed in several ways in statistics, including presentation of the total range of data values, interquartile range (IQR), variance, and standard deviation. A simple useful parameter to compare variability between different datasets is the coefficient of variation (CV) or the relative standard deviation, which is calculated by dividing the standard deviation to the mean. Another useful parameter is the standard scores, usually called z-scores, which express how far is certain data from the mean and are calculated as a ratio between the difference of analyzed data and the mean from one side and the standard deviation, form the other side.

Although analyzed results of specified calculations may be distributed around the mean, there might be some data items, which are far apart from the mean addressed as outliers, causing irrelevant conclusions if there are corruptions in the data input or performed calculations. The guidelines to remove outliers include at least IQR and z-scores methods. The IQR method divides the data range in quartiles of equal size, expressing the acceptable distribution in the middle 50% of the dataset including values in the median between the third quartile (higher than 75^{th} percentile) and the first quartile (lower than 25^{th} percentile) and specifying the other data items to be outliers. The Z-score method detects outliers after data centering around zero and rescaling if the data items are too far from zero with a threshold value of 3 or −3.

### Glucose Regulation Classes

Blood glucose level is measured by fasting plasma blood glucose levels as an instantaneous glucose indication or by glycosylated hemoglobin HbA1C (often referred to as A1C) that is an indicator of the degree of glycemic control (usually referred to as a measure of the ability to control the blood sugar over a period of about 2 or 3 months). The HbA1C value is measured in percentage of hemoglobin (a protein in red blood cells that carries oxygen) that is glycated (coated with sugar).

The patients are classified into the following three classes considering the ability to regulate the glucose level or based on fasting plasma blood glucose level (ADA, 2020).
ND class, meaning non-diabetic or pre-diabetes patients that had no problems with the regulation of glucose level if the measured values of HbA1C are <6.5% (47 mmol mol) or fasting plasma glucose level<6.9 mmol L (125 mg dL),
GD class, a subset of diabetic patients with good regulation of glucose level, by therapies including food, medicaments, or insulin, if the measured values of HbA1C are <6.5% (47 mmol mol) or fasting plasma glucose level<6.9 mmol L. (125 mg dL), and
BD class, a subset of diabetic patients with bad regulation of glucose level, meaning that their treatment with food, medicaments, or insulin is not sufficient if the measured values of HbA1C are >=6.5% (47 mmol mol) or fasting plasma glucose level>=6.9 mmol L (125 mg/dL).

Continuous glucose monitoring (CGM) systems realized mostly as minimally invasive methods enabled frequent plasma glucose measurements. AGP was promoted to visualize the data presentation of CGM, including several glucose statistical metrics, such as percentage of time per day within a target glucose time in range (TIR), time below target glucose range (TBR) within the hypoglycemic periods, and time above target glucose range (TAR) within the hyperglycemic periods. The mean value of all CGM measurements within a period of time give an overall impression of average ability to control the glucose level, indicating the average time spent in the corresponding range.

Other metrics that can express the glucose profile include statistical measures M-value, mean of daily difference, continuous overall net value, etc. Glycemic variability (GV) refers to changes and oscillations in blood glucose levels throughout the day is also used to express the glucose profile and fluctuations on different days. The methods to calculate short-term GV include SD and CV of all CGM glucose measurements, and the mean amplitude of glycemic excursions (MAGE). There is no gold-standard method to calculate GV, although a lot of research is ongoing.

### DETAILED DESCRIPTION OF THE EMBODIMENTS

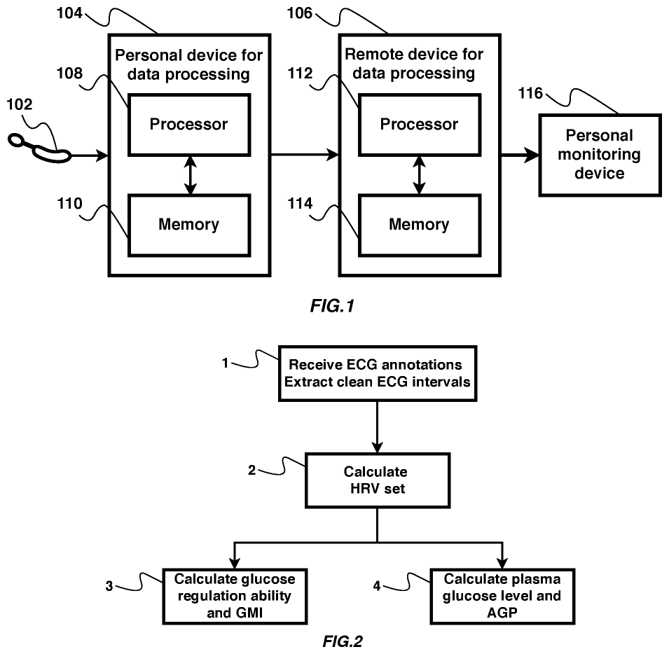

FIG. 1 shows a system for blood glucose monitoring. The system for blood glucose monitoring comprises a device for ECG sensing 102 and a personal device for data analysis 104.

The ECG sensing device 102 may be worn by a mammal on a chest. The ECG sensing device 102 may be built as a small wearable patch that uses a small internal battery to enable long-term ECG measurements in terms of one or more days. To provide longer battery life, this device does not process extended calculations or store data, rather, it sends all sensed data to the personal device for data analysis 104. The ECG sensing device may be coupled to the personal device for data analysis 104 with a communication link. In various embodiments, the communication link may use various communication network technologies, including Bluetooth, infrared, ultrasound, Wi-Fi, ADSL, or any other similar radio communication technology.

The ECG sensing device 102 may send ECG samples to the personal device for data analysis 104. The personal device for data analysis 104 comprises a processor 108 and a memory 110. The personal device for data analysis 104 may be configured to receive ECG samples from the ECG sensing device 102 and to store them in the memory 110. The processor 108 may be configured to perform beat detection, beat classification, and annotation sharing.

In one embodiment, the personal device for data analysis 104 may be coupled to a remote device for data analysis 106 via a communication link. The remote device for data analysis 106 comprises a processor 112 and a memory 114. The remote device for data analysis 106 is coupled to receive ECG data samples and annotations from the personal device for data analysis 104 and to store received ECG data in memory 114. The processor 112 may be configured to run an algorithm to perform extended beat detection and classification. The processor 112 may be configured to run an algorithm to perform detection of the ability to regulate glucose and glucose profile. In one embodiment the remote device for data analysis 106 may be implemented as a cloud-based system. In another embodiment, the remote device for data analysis 106 may be implemented as a shared data center or a similar processing and communication network environment.

In one embodiment, the remote device for data analysis 106 may be coupled to a personal computer 116. In another embodiment, the remote device for data analysis 106 may be coupled to a smart device or similar device 116 capable to provide remote monitoring to doctors and caregivers.

To detect the ability for glucose regulation and glucose profile based on received beat annotations the remote device for data analysis 106 is configured to
1) eliminate beats succeeding the beat to be eliminated with standard methods;
2) eliminate large beat-to-beat interval changes caused by noise corrupted signal;
3) eliminate short sequences of N beats;
4) extract clean ECG segments;
5) use the individual and concatenated methods to calculate an extended set of HRV with distribution coverage factor over the analyzed window;
6) calculate a collection of extended HRV parameters by the sliding window approach on ECG measurements;
7) eliminate the outliers in the calculated collection and forming an aggregation of HRV collections.

The personal device for data analysis 104 may be configured to eliminate the ectopic and premature beats, and missing, extra, or misaligned beat detections. The personal device for data analysis 104 may be configured to eliminate the beat following the eliminated beat. The reason is due to the way the QRS detector detects the beats, and the performance, which was interrupted by the occurrence of the eliminated beats. It usually needs one extra beat to start precise detection. These beats are annotated as dirty beats.

To ensure proper beat detection in cases of noise corrupted ECGs or detection of artifacts the personal device for data analysis 104 may be configured to eliminate those beats which occurrence indicates sudden heart rate changes by checking if the relative proportion between the analyzed beat-to-beat interval and its predecessor is higher than a threshold value ThrA. In case the change is significant, the beats in the analyzed beat-to-beat interval and the beat that succeeds them are marked as dirty beats.

To ensure a sufficient HRV behavior the personal device for data analysis 104 may be configured to eliminate those sequences of N beats that contain less than ThrB beats. These small sequences of N beats and the beat that succeeds them are also marked as dirty beats.

The personal device for data analysis 104 may be configured to mark of dirty intervals with a starting point in the middle of the RR interval preceding a dirty beat and ending in the middle of the RR interval that succeeds the last dirty beat in the sequence. In addition, the personal device for data analysis 104 may be configured to join neighboring dirty intervals into larger ones. The ECG intervals that remain unmarked as dirty are annotated as “clean ECG intervals”

A particular ECG measurement time frame may be classified as:
Short-term ECG measurements that include intervals from 10 seconds up to 5 minutes,
Medium-term ECG measurements that include intervals larger or equal to 5 minutes up to 1 hour,
Long-term ECG measurements that include intervals larger or equal to 1 hour up to 12 hours, and
Extra long-term ECG measurements that include intervals larger or equal to 12 hours up to 48 hours.

The personal device for data analysis 104 may use a sliding window approach to calculate ECG measurement windows with a specified time frame, wherein the sliding window approach starts with different offsets to the beginning of the complete EGC measurement. The analysis may continue with windows that contain at least one clean ECG intervals. In one embodiment, the personal device for data analysis 104 may calculate HRV for each clean ECG segment separately and then may use the average value as HRV for that window. This approach may be identified as an individual approach. In another embodiment, the personal device for data analysis 104 may calculate the HRV for the analyzed ECG measurement window with the concatenated approach that joins (combines) the clean ECG intervals into one larger interval and may use the standard calculation of the HRV. The mathematical operations used for the individual or concatenated approaches may be calculating an arithmetic average, harmonic or geometrical mean, calculating standard deviation, or any other statistical measure.

The personal device for data analysis 104 may be configured to detect a coverage factor over the whole ECG measurement window to verify the distribution spread over the ECG parts. The personal device for data analysis 104 is configured to divide the ECG measurement window in Nc parts and to check if the clean ECG segments are (distributed) spread in the majority of these parts (more than half of Nc).

The personal device for data analysis 104 may use an extended set of HRV parameters correspondingly annotated by A, C, S in addition to the set of standard HRV, wherein A_SDNN calculates the SDNN HRV parameter using the specified individual method with a coverage factor of clean ECG intervals, C_SDNN calculates the SDNN HRV parameter using the specified concatenated approach of clean ECG intervals satisfying the coverage factor condition, and S_SDNN calculates the SDNN HRV parameter as a standard deviation of clean ECG intervals satisfying the coverage factor condition.

A collection of extended HRV sets may be calculated for any of the specified ECG measurement time intervals, including short, medium, long, and extra-long term. An aggregation of collections of extended HRV sets may also be calculated for short and medium-term measurements specifying the offset prior to the analyzed time moment. The sliding window method may be used to traverse all window sizes and offsets with respect to the analyzed moment. To reveal more reliable results, the outliers may be removed by IQR and/or Z-scores method.

To estimate the ability to control the glucose levels based on HRV the personal device for data analysis 104 may:
1) include a set of threshold values ThrC₁, ThrC₂, . . . , ThrCₙ for HRV parameters HRV₁, HRV₂, . . . , HRV, that include at least A_SDNN, A_RMSSD, S_RMSSD, and NN50 measured for extra-long term ECG measurements with a coverage factor of ECG parts;
2) estimate that the patient cannot regulate the glucose level if all calculated HRV values HRV₁, HRV₂, . . . , HRV, are smaller than corresponding thresholds ThrC₁, ThrC₂, . . . , ThrCₙ;
3) estimate that the patient can regulate the glucose level if all calculated HRV values HRV₁, HRV₂, . . . , HRV, are greater than corresponding thresholds ThrC₁, ThrC₂, . . . , ThrCₙ;
4) estimate that the patient cannot regulate the glucose level if the majority of calculated HRV values HRV₁, HRV₂, . . . , HRV, are smaller than corresponding thresholds ThrC₁, ThrC₂, . . . , ThrCₙ;
5) estimate that the patient can regulate the glucose level if the majority of calculated HRV values HRV₁, HRV₂, . . . , HRV, are not smaller than corresponding thresholds ThrC₁, ThrC₂, . . . , ThrCₙ;
6) calculate the GMI based on calculated values of specified HRV parameters by a mathematical function of their combination and corresponding weighted factors to HRV parameters, using a regression function, or other mathematical or computer science method, including NN, ML, or DL. In this context, note that the function for lower values may be different from the function of higher values.

To estimate the instantaneous plasma glucose level based on HRV the personal device for data analysis 104 may:
1) specify a set of HRV parameters HRV₁, HRV₂, . . . , HRV, that include at least A_SDNN, A_RMSSD, S_RMSSD, and NN50 measured for short and medium-term ECG measurements for a predefined time interval; with an offset from the start of the analyzed window up to the predefined time interval prior to the start moment with a coverage factor of ECG parts specified in this document: in one embodiment, the predefined time interval may be 1 hour;
2) calculate the instantaneous plasma glucose level based on calculated short and medium-term values of the specified HRV parameters by a regression function or similar method, including NN, ML, or DL.
3) calculate the glucose profile with details on AGP, GV, TIR, TBR, TAR, MAGE

The personal device for data analysis 104 may act as an automated monitoring and reporting software agent. The personal device for data analysis 104 may check the ECG signal to estimate the blood glucose level and may be used to generate reports containing the AGP, period glucose profiles, and glucose statistics and targets, as suggested by Standards of Medical Care in Diabetes, 2020 Abridged for Primary Care Providers, (ADA, 2020) which is characteristic of CGM systems using minimally invasive methods. The personal device for data analysis 104 may calculate the glucose profile from data obtained by continuous ECG monitoring. In one embodiment of the personal device for data analysis 104 may work continuously, wherein the personal device for data analysis 104 may be fed by non-invasive wearable ECG sensors.

FIG. 2 shows a method for blood glucose monitoring. The method for blood glucose monitoring is coupled to receive an array of annotations for ECG strips (Block 1). The array of annotations for ECG strips is processed to mark the dirty intervals and extract clean ECG intervals (Block 1). The method for blood glucose monitoring then calculates the set of HRV parameters and aggregation of HRV collections (block 2). The method for blood glucose monitoring then uses calculated HRV values to make threshold decisions to calculate glucose regulation ability (block 3). The method detects the qualitative value of ability to regulate the glucose level and calculates the quantitative value of GMI as equivalent to the HbA1C (block 3). The method for blood glucose monitoring uses calculated HRV values to calculate the set of plasma glucose levels and glucose profile metrics specified in AGP, including GV, TIR, TAR, TBR, MAGE (block 4).

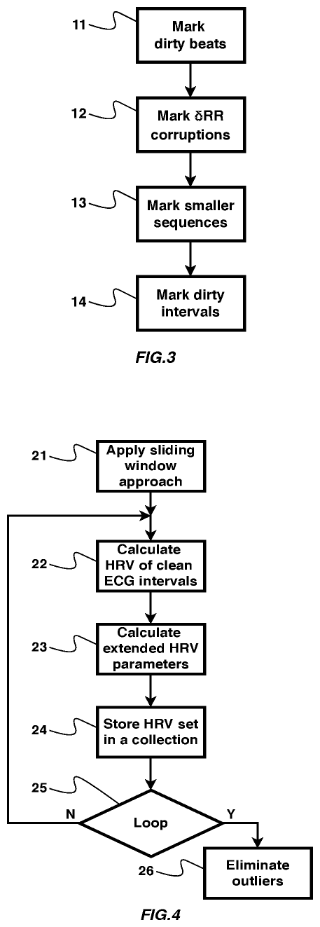

FIG. 3 shows a method for extracting clean ECG signals. The method for extracting clean signals receives ECG annotations and starts by marking dirty beats and the beats that succeed them (block 11). The method marks a dirty beat if it does not belong to the N class (either belongs to the V, S, F or Q class) or is identified as an artifact or belongs to a segment identified as noise or unreadable data, and also marks a dirty beat to be the normal beat that succeeds the identified dirty beat (block 11). The method for extracting clean ECG signals then marks dirty beats where the ratio δRR of the successive difference of NN intervals and the analyzed NN interval is beyond the threshold (block 12). The method analyzes three consecutive N beats and calculates the absolute value of the successive difference between the first and second RR interval; and the ratio ORR of the successive difference and second RR interval. If δRR>ThrA then the beats associated to the second RR interval are marked as dirty. FIG. 10 illustrates a case where δRR₃=|(RR₃−RR₂)|/RR₃>ThrA and Nc and ND are marked as dirty beats (under the interval identified by z). The method analyzes the sequences of consecutive N beats, which remain after marking the dirty beats, and counts the number of N beats within these sequences. If the number of N beats in these sequences is smaller than ThrB, then these sequences are also marked as dirty. FIG. 9 illustrates these beats that belong to the sequences identified as y for ThrB=6. The method for extracting clean ECG signals then marks dirty intervals that include dirty beats and smaller intervals not marked as dirty if they contain a smaller number of beats than a threshold value (block 13). The method analyzes the sequences of consecutive N beats, which remain after marking the dirty beats and counts the number of N beats within these sequences. If the number of N beats in these sequences is smaller than ThrB, then these sequences are also marked as dirty. FIG. 9 illustrates these beats that belong to the sequences identified as y for ThrB=6. The method for extracting clean ECG signals then marks dirty intervals and extracts clean ECG intervals (block 14). The method marks the dirty intervals, by the following procedure: it marks a start of a dirty interval from the middle of the RR interval between two successive beats if the successor beat is identified as dirty and the predecessor is not. The end of the dirty interval is marked in the middle of the RR interval after the last dirty beat in the sequence. FIG. 9 illustrates the marking of a dirty interval by x for applying the calculation block 11, by y for applying the calculation block 13, and FIG. 10 by z for applying the calculation block 12. All remaining intervals after performing blocks 11, 12, 13, and 14 are “clean ECG intervals”.

FIG. 4 shows a method for calculating HRV parameters as an aggregation of HRV collections. The method starts when it receives clean ECG intervals. The method applies the sliding window approach (block 21), which specifies a repetitive loop to generate extended HRV sets for a specific window frame (depending on the time duration from 30 seconds to 24 h specified by short, medium, long, and extra-long term measurements) and offset from the analyzed window (within an interval from the start of the analyzed window up to 1 hour prior to analyzed window start). The method calculates HRV parameters of clean ECG intervals (block 22). Then extended HRV parameters are calculated by dividing the analyzed window into Ne parts, calculating the coverage factor, applying the individual or concatenated approaches to calculate any of the following operations, arithmetic, harmonic or geometric means, or standard deviation on the HRV of clean ECGs (block 23). Note that a relevant result is released only in case the coverage factor shows that the majority of ECG parts contain at least one clean ECG.

Values of the calculated extended set of HRV for each window and offset are stored in a memory (Block 24). The method tests if all sets are calculated (block 25). If not all sets are calculated (N branch of block 25), the method repeats blocks 22, 23, and 24 for another combination of a window or offset size. This results in a collection of HRV sets when all the sizes are processed.

If all sets are calculated (Y branch of block 25), the method eliminates the outlier values (block 26), which differs from the average behavior and range of possible values, (located outside the region formed around the average value and standard deviation of the analyzed HRV) or is specified as an outlier by the IQR or Z score statistical methods. This final step finishes with an aggregation of HRV collections for different window and offset sizes, and different HRV parameters calculated by the individual or concatenated approaches for clean ECGs satisfying the coverage factor condition for the presence of clean ECG intervals in most ECG parts.

Referring now to FIG. 2, the aggregation of HRV collections, resulting from block 2 are provided to blocks 3 and 4 which can be executed concurrently to estimate the ability to regulate the glucose, the equivalent value of HbA1C value and the plasma glucose level.

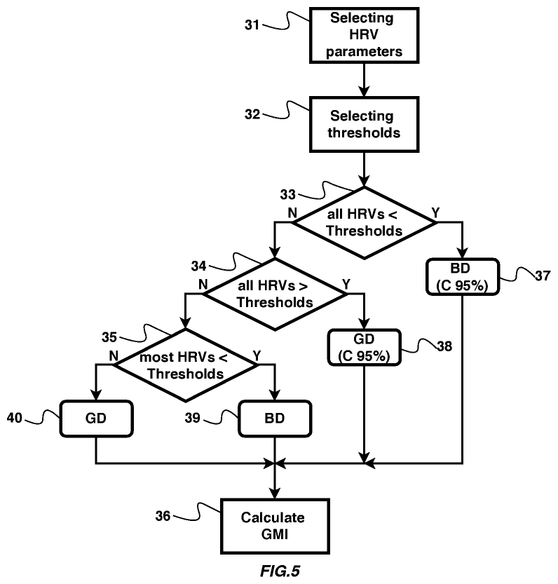

FIG. 5 shows a method for calculating glucose regulation ability and GMI. The method begins when it receives aggregation of HRV collections. Aggregation of HRV collections are processed in order to select a set of HRV parameters, which consists of long term and extra-long term ECG measurements (block 31). A set of thresholds is selected based on previous experience, calibration, customization, and update (block 32). Then the calculated HRVs are compared to previously specified thresholds (block 33). If all HRVs are smaller than the thresholds (Y branch of block 33), than the method concludes that the ability to regulate the glucose is bad (class is BD) with at least 95% confidence (block 37). If all HRVs are not smaller than the thresholds (N branch of block 33), then the method tests if all HRVs are greater than the thresholds (block 34). If all HRVs are greater than the thresholds (Y branch of block 34), then the method concludes that the ability to regulate the glucose is good (class is GD) with at least 95% confidence (block 38). If all HRVs are not greater than the thresholds (N branch of block 34), then the method tests if the majority of HRVs are smaller than the threshold (block 35). If the majority of HRVs are smaller than the threshold (Y branch of block 35), the method decides bad ability to regulate glucose (class is BD) (block 39). If the majority of HRVs are not smaller than the threshold (N branch of block 35), the method decides good ability (class is GD) (block 40).

The method processes the selection of HRVs in order to calculate the GMI as equivalent to HbA1C (block 36). The calculations include a combination of the input parameters with corresponding weighting factors, using a regression function, and or other mathematical or computer data science method, including NN, ML or DL. Some embodiments may include different calculation functions within the regions determined by good and bad ability to regulate the glucose levels.

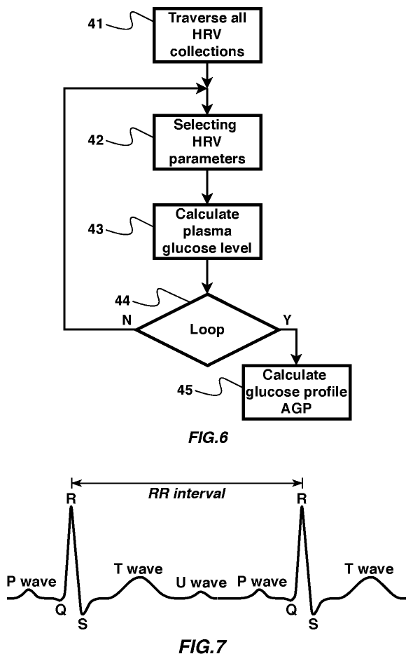

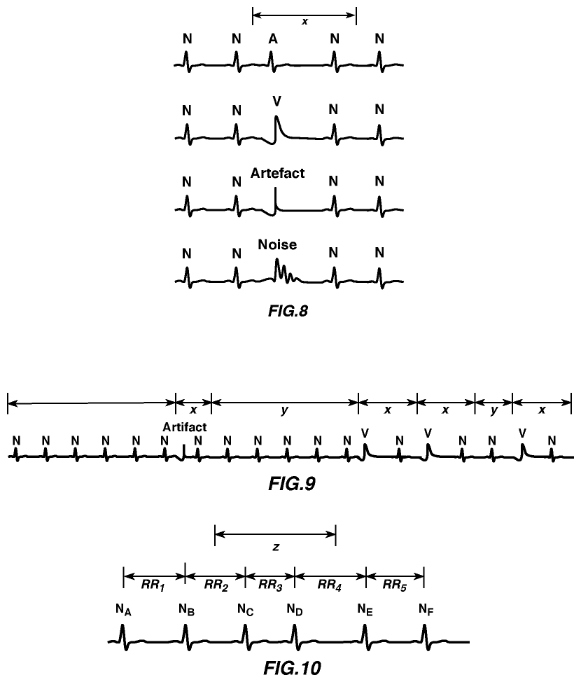

FIG. 6 shows a method for calculating the set of detected plasma glucose levels and a set of metrics that determine the AGP, including GV, TIR, TAR TBR, and MAGE from the aggregation of HRV collections. The method traverses all HRV collections to output an analyzed HRV collection for processing (block 41). Specific HRV parameters are selected (block 42) and calculations that result in plasma glucose values are performed (block 43).

The method selects an HRV collection for short-term and medium-term ECG measurements with different offset to the analyzed window start (block 42). The method processes the obtained selection of HRVs within the analyzed collection and calculates the plasma glucose level using a regression function, and or other mathematical or computer data science method, including NN, ML, or DL on a combination of the input parameters with corresponding weighting factors (block 43).

The method tests if all the windows with a corresponding HRV collection are traversed (block 44). If not all windows are traversed; the method selects an HRV collection (N branch of 44) and repeats blocks 42 and 43. The traversing activities result in a set of plasma glucose levels for the analyzed time period. If all windows are traversed (Y branch of 44), the method calculates the relevant AGP metrics, including GV, TIR, TBR, TAR, and MAGE (block 45). Some embodiments may include different calculation functions within specific regions, such as those determined as good and bad ability to regulate the glucose levels.

1. A system for non-invasive monitoring glucose level comprising a device for ECG sensing and a personal device, wherein the personal device is configured to
receive ECG samples from the device for ECG sensing;
perform beat detection, beat classification, and annotation sharing;
extract a clean ECG signal;
calculate an extended set of HRV parameters;
calculate an aggregation of HRV collections with predefined window sizes and offsets;
apply a set of threshold decisions to detect ability to regulate a predefined glucose level;
calculate an equivalent to HbA1C and plasma glucose levels;
form reports containing AGP and achieved glucose statistics.

2. The system in claim 1, wherein the personal device is configured to extract the clean ECG signal using marking of dirty beats, wherein the personal device is configured to
identify first beats that do not belong to a normal N class and second beats that succeed said first beats;
identify third beats, wherein said third beats are identified as artifacts or segments with identified noise;
identify fourth beats that succeed third beats;
identifying fifth beats where beat-to-beat interval differs from a previous beat-to-beat interval for a factor larger than a predefined threshold;
identifying sixth beats that succeed the fifth beats;
identifying seventh beats that are not marked as dirty, wherein a longest continuous sequence is smaller than a predefined threshold.

3. The system in claim 1 wherein the personal device is configured to extract the clean ECG signal using marking of dirty intervals, wherein the personal device is configured to
mark a start of a dirty interval in a middle of a beat-to-beat interval, wherein a first beat belongs to a normal N class, wherein a second beat is marked as a dirty beat;
mark the start of the dirty interval in a location after a T wave of a first beat and before a P wave of a second beat;
mark an end of the dirty interval in the middle of the beat-to-beat interval, where the first beat is marked as the dirty beat, and the second beat belongs to the normal N class;
mark the end of the dirty interval in the location after the T wave of the first beat and before the P wave of the second beat.
extract the clean ECG intervals, wherein the clean ECG intervals are intervals that are not marked as dirty intervals.

4. The system in claim 1, wherein the personal device is configured to calculate the extended set of HRV parameters using individual or concatenated approaches of clean ECG intervals, wherein the personal device is configured to
calculate a set of HRV parameters on the clean ECG intervals within an analyzed ECG measurement window;
calculate statistical operations on the set of HRV parameters calculated on clean ECG intervals;
concatenate clean ECG intervals from the analyzed ECG measurement window into a long ECG interval;
calculate the HRV on the long ECG interval.

5. The system in claim 1, wherein the personal device is configured to calculate the aggregation of HRV collections using a sliding window approach satisfying the coverage factor condition of including the clean ECG intervals within the analyzed ECG measurement, wherein the personal device is configured to
specify a repetitive procedure that calculates a collection of HRVs for predefined ECG window sizes with short, medium, long and extra-long term ECG measurements;
specify a repetitive procedure that calculates a collection of HRVs for predefined offset in respect to an analyzed time moment that includes ECG window sizes with short and medium term ECG measurements corresponding to a predefined first duration and a predefined second duration;
calculate a minor coverage factor as a number of ECG parts that contain at least one clean ECG interval within an analyzed ECG part;
calculate a major coverage factor as the number of ECG parts where a time duration of clean ECG intervals within a first ECG part cover more than half of a time duration of the analyzed ECG part;
specify a coverage factor condition by identifying a relevant HRV collection if a minor coverage factor or the major coverage factor include a majority of ECG parts if a minor coverage factor or the major coverage factor are more than a half of the number of ECG parts;
eliminate a HRV from a collection set if the HRV differs from an average behavior and a range of possible values that are outside a region formed around an average value and a standard deviation of an analyzed HRV;
eliminate the HRV from the collection set by applying an IQR, Z score.

6. The system in claim 1, wherein the personal device is configured to apply set of threshold decisions for detecting the ability to regulate the glucose level, wherein the personal device is configured to
select a specific set of HRV parameters from an aggregation of HRV collections;
classify a bad ability to regulate glucose levels with a predefined confidence if all HRVs are smaller than a plurality of thresholds;
classify a good ability to regulate glucose levels with a predefined confidence if all HRVs are higher than the plurality of thresholds;
determine a number of results if a number of selected HRVs are smaller than the plurality of thresholds and classifying the bad ability to regulate glucose levels if the number of results is smaller than the number of selected HRVs.

7. The system in claim 1, wherein the personal device is configured to calculate the equivalent to HbA1C as a value that expresses the two-month average ability to regulate glucose is accomplished by calculating a regression function or any other mathematical, computer or data science functional method of selected dominant HRVs, wherein the personal device is configured to
select a set of dominant long and extra-long term HRVs that impact overall ability to regulate glucose level;
specify a processing procedure based on applying weighting factors to selected HRVs.

8. The system in claim 1, wherein the personal device is configured to calculate the equivalent to an instantaneous plasma glucose level by calculating a regression function or any other mathematical, computer or data science functional method of selected dominant HRVs, wherein the personal device is configured to
select an aggregation of dominant short and medium term HRV collections with different window sizes and different offsets to the analyzed time moment that impact the instantaneous plasma glucose level;
specify a processing procedure based on applying weighting factors to selected HRVs.

9. The system in claim 1, wherein the personal device is configured to form the reports containing AGP and glucose statistics by selecting a specific time frame and calculating methods to calculate average glucose behavior and variability indexes and coefficients, wherein the personal device is configured to
select a time frame to analyze instantaneous plasma glucose levels;
calculate the average glucose behavior;
calculate an average behavior of glucose statistics and an ambulatory glucose profile, including a glycemic control index, a glucose management index, a glucose variability, a coefficient of glucose variation (CV), a time spent in normal range, a time spent below range, a time spent after range, and a mean amplitude of glycemic excursions (MAGE).

10. A method of detecting the glucose level by non-invasive method comprising:
analyzing ECG annotations;
extracting a clean ECG interval;
calculating an extended set of HRV parameters;
calculating an aggregation of HRV collections with predefined window sizes and offsets;
applying a set of threshold decisions to detect ability to regulate a predefined glucose level;
calculating an equivalent to HbA1C and plasma glucose levels;
forming reports containing AGP and achieved glucose statistics.

11. The method in claim 10 wherein the extracting step is accomplished by using marking of dirty beats, wherein using marking of dirty beats comprising
identifying first beats that do not belong to a normal N class and second beats that succeed said first beats;
identifying third beats identified as artifacts or segments with identified noise;
identifying fourth beats that succeed second beats;
identifying fifth beats where beat-to-beat interval differs from a previous beat-to-beat interval for a factor larger than a predefined threshold;
identifying sixth beats that succeed the fifth beats;
identifying seventh beats that are not marked as dirty, wherein a longest continuous sequence is smaller than a predefined threshold.

12. The method in claim 10 wherein the extracting step is accomplished by using marking dirty intervals, wherein marking of dirty intervals comprising
marking a start of a dirty interval in a middle of a beat-to-beat interval, wherein a first beat belongs to a normal N class, wherein a second beat is marked as a dirty beat;
marking the start of the dirty interval in a location after a T wave of the first beat and before a P wave of the second beat;
marking an end of the dirty interval in the middle of the beat-to-beat interval, where the first beat is marked as the dirty beat, and the second beat belongs to the normal N class;
marking the end of the dirty interval in the location after the T wave of the first beat and before the P wave of the second beat.
extracting the clean ECG intervals, wherein the clean ECG intervals are intervals that are not marked as dirty intervals.

13. The method of claim 10 wherein the step of calculating the extended set of HRV parameters is accomplished by using individual or concatenated approaches of clean ECG intervals comprising
calculating a set of HRV parameters on clean ECG intervals within an analyzed ECG measurement window;
calculating statistical operations on the set of HRV parameters calculated on clean ECG intervals;
concatenating clean ECG intervals from the analyzed ECG measurement window into a long ECG interval;
calculating the HRV on the long ECG interval.

14. The method of claim 10 wherein the step of calculating the aggregation of HRV collections is accomplished by using a sliding window approach satisfying the coverage factor condition of including the clean ECG intervals within the analyzed ECG measurement comprising
specifying a repetitive procedure that calculates a collection of HRVs for predefined ECG window sizes with short, medium, long and extra-long term ECG measurements;
specifying a repetitive procedure that calculates a collection of HRVs for predefined offset in respect to an analyzed time moment that includes ECG window sizes with short and medium term ECG measurements corresponding to a predefined first duration and a predefined second duration;
calculating a minor coverage factor as a number of ECG parts that contain at least one clean ECG interval within an analyzed ECG part;
calculating a major coverage factor as the number of ECG parts where a time duration of clean ECG intervals within a first ECG part cover more than half of a time duration of the analyzed ECG part;
specifying a coverage factor condition by identifying a relevant HRV collection if the minor coverage factor or the major coverage factor include a majority of ECG parts if the minor coverage factor or the major coverage factor are more than a half of the number of ECG parts;
eliminating a HRV from a collection set if the HRV differs from an average behavior and a range of possible values that are outside a region formed around an average value and a standard deviation of an analyzed HRV;
eliminating the HRV from the collection set by applying an IQR, Z score.

15. The method of claim 10 wherein the step of applying set of threshold decisions for detecting the ability to regulate the glucose level comprising
selecting a specific set of HRV parameters from an aggregation of HRV collections;
classifying a bad ability to regulate glucose levels with a predefined confidence if all HRVs are smaller than a plurality thresholds;
classifying a good ability to regulate glucose levels with a predefined confidence if all HRVs are higher than the plurality thresholds;
determining a number of results if a number of selected HRVs are smaller than the plurality thresholds and classifying the bad ability to regulate glucose levels if the number of results is smaller than the number of selected HRVs.

16. The method of claim 10 wherein the step of calculating the equivalent to HbA1C as a value that expresses the two-month average ability to regulate glucose is accomplished by calculating a regression function or any other mathematical, computer or data science functional method of selected dominant HRVs comprising
selecting a set of dominant long and extra-long term HRVs that impact overall ability to regulate glucose level;
specifying a processing procedure based on applying weighting factors to selected HRVs.

17. The method of claim 10 wherein the step of calculating the equivalent to instantaneous plasma glucose level is accomplished by calculating a regression function or any other mathematical, computer or data science functional method of selected dominant HRVs comprising
selecting an aggregation of dominant short and medium term HRV collections with different window sizes and different offsets to the analyzed time moment that impact the instantaneous plasma glucose level;
specifying a processing procedure based on applying weighting factors to selected HRVs.

18. The method of claim 10 wherein the step of forming the reports containing AGP and glucose statistics is accomplished by selecting a specific time frame and calculating methods to calculate average glucose behavior and variability indexes and coefficients comprising
selecting a time frame to analyze instantaneous plasma glucose levels;
calculating the average glucose behavior;
calculating an average behavior of glucose statistics and ambulatory glucose profile, including a glycemic control index, a glucose management index, a glucose variability, a coefficient of glucose variation (CV), a time spent in normal range, a time spent below range, a time spent after range, and a mean amplitude of glycemic excursions (MAGE).
